(19) United States
(12) Patent Application Publication
(10) Pub. No.: US 2018/0177720 A1
(43) Pub. Date: Jun. 28, 2018

(54) Chewable Composition and Delivery Pouch

(71) Applicant: Nicholas Cooper, Manila, AR (US)

(72) Inventor: Nicholas Cooper, Manila, AR (US)

(21) Appl. No.: 15/854,041

(22) Filed: Dec. 26, 2017

Related U.S. Application Data
(60) Provisional application No. 62/438,570, filed on Dec. 23, 2016.

(51) Int. Cl. A61K 9/00; A61K 31/352; A61K 31/05; A61K 36/185; A61K 47/46

(52) U.S. Cl. CPC A61K 9/0056; A61K 31/352; A61K 47/46; A61K 36/185; A61K 31/05

(57) ABSTRACT

A chewable delivery pouch including a chewable composition containing cannabis. An interior volume of the chewable pouch is filled with a composition having a quantity of mint, coconut oil and cannabis plant extracts. In some embodiments, the coconut oil is replaced with another bedewer, such as vegetable glycerin. The chewable delivery pouch allows a user's saliva and dissolved molecules in the sputum to pass between the interior volume of the pouch and a user's mouth.

### CROSS REFERENCE TO RELATED APPLICATIONS

This application claims the benefit of U.S. Provisional Application No. 62/438,570 filed on Dec. 23, 2016. The above identified patent application is herein incorporated by reference in its entirety to provide continuity of disclosure.

### BACKGROUND OF THE INVENTION

The present invention relates to a chewable composition incorporating cannabis plant extracts and means of delivery. Specifically, the present invention provides a chewable pouch containing a homogenous mixture of a quantity of mint, a quantity of a bedewer and a quantity of a cannabis plant extract.

The cannabis genus of plants, namely Cannabis sativa, is known and desired for the production of psychoactive compounds. These psychoactive compounds have numerous medical and recreational benefits, for example appetite stimulation in HIV/AIDS patients, reduction of nausea and vomiting during chemotherapy and the treatment of chronic pain. The beneficial compounds of cannabis are primarily constituent cannabidiols (CBD) and tetrahydrocannabinol (THC).

Currently, a person who seeks the effects cannabis must either smoke the cannabis or ingest cannabis extracts by eating or drinking an infused substance. Thus, a chewable product such as a product similar to snuff, dip or other forms of chewable tobacco is desired to provide a person with an alternate less-invasive means of therapy.

Several devices and compositions have been proposed to provide this alternative means of cannabis consumption. One strategy provides a cannabis infused chewing composition containing ground cannabis, cannabis extracts and a quantity of flavor enhancer. This cannabis infused chewing composition, however, fails to provide a pouch to keep the chewable composition concentrated while in-use and, also, fails to provide a mint as being the main constituent of the chewable composition.

### SUMMARY OF THE INVENTION

In view of the foregoing disadvantages inherent in the known types of chewable compositions containing cannabis now present in the known art, the present invention provides a chewable composition containing cannabis and pouch for containing the same wherein the same can be utilized for providing convenience to a user when acquiring the therapeutic effects of cannabis. The present system comprises a chewable delivery pouch configured to allow saliva and dissolved molecules therein to pass between a mouth and an interior volume of the pouch. An interior volume of the chewable delivery pouch contains a cannabis incorporated chewable composition. The chewable composition is further comprised of a quantity of mint, a quantity of a bedewer and a quantity of a cannabis plant extract.

One object of the present invention is to provide a chewable composition containing cannabis extracts that is presented in a chewable pouch, whereby the user can chew on the pouch and receive the therapeutic benefits of the chewable composition contained therein.

Another object of the present invention is to provide a chewable composition containing cannabis extracts that leaves a fresh and minty taste in a user's mouth.

Yet another object of the invention is to provide a chewable composition containing cannabis extracts that do not contain the psychoactive THC molecule but still contain a therapeutically beneficial CBD isolate.

Other objects, features, and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the delivery pouch with a cut-out portion showing an embodiment of the chewable composition therein.

### DETAILED DESCRIPTION OF THE INVENTION

Reference is made herein to the attached drawings. Like reference numerals are used throughout the drawings to depict like or similar elements of the chewable composition and delivery pouch. The figures are intended for representative purposes only and should not be considered to be limiting in any respect. Unless specifically limited to a single unit, “a” is intended to be equivalent to “one or more” throughout the present disclosure.

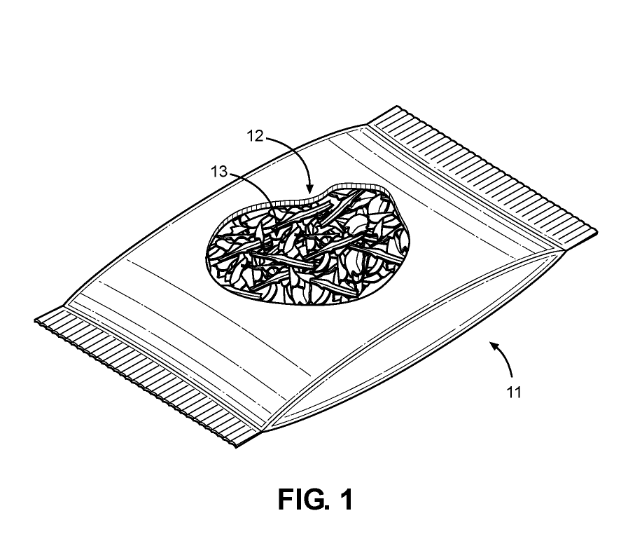

Referring now to FIG. 1, there is shown a perspective view of an embodiment of the delivery pouch with a cut-out portion showing an embodiment of the chewable composition sequestered therein. The chewable composition and delivery pouch comprises a chewable delivery pouch 11 having an interior volume 12. Within the interior volume 12 of the chewable delivery pouch 11, is a chewable composition 13. In this illustrated embodiment, the chewable composition 13 is comprised of a quantity of mint, a quantity of a bedewer and a quantity of a cannabis plant isolate in the forms of tetrahydrocannabinol isolate and cannabidiol isolate. Also, in certain embodiments, the chewable composition 13 also comprises a quantity of stevia extract. One such embodiment of the chewable composition 13 consists of 500 mg of mint, 200 mg of stevia extract, 10 mg of bedewer, 10 mg of tetrahydrocannabinol isolate and 10 mg of cannabidiol isolate.

The chewable delivery pouch 11 is made of a soft, semipermeable material. The semipermeable nature of the chewable delivery pouch 11 allows saliva and dissolved compounds therein to pass through the chewable delivery pouch 11, thereby effectuating the translocation of saliva and dissolved compounds from the chewable composition 13 between the interior volume 12 of the chewable delivery pouch 11 and a mouth of a user. In some embodiments, the chewable delivery pouch 11 is made of a filter paper. In other embodiments, the chewable delivery pouch 11 is made from cotton, silk or synthetic polymer fiber.

The quantity of mint in the chewable composition 13 can be either a quantity of spearmint or a quantity of peppermint, depending on the embodiment. The mint serves to freshen the breath of a user. In the present embodiment, the mint constitutes 68.5 percent of the total mass of the chewable composition 13. In other embodiments, the mint comprises an amount in the range of 50% to 90% of the total mass of the chewable composition 13.

The quantity of bedewer, in one embodiment of the chewable composition 13, is a quantity of coconut oil. In another embodiment, a quantity of vegetable glycerin serves as the bedewer. The bedewer is utilized to moisten the chewable composition 13; and thereby, providing a more enjoyable chewing experience than chewing on a dry leafy composition. In the present embodiment, the bedewer comprises 1.4% of the total mass of the chewable composition 13. In other embodiments, the bedewer comprises an amount in the range of 1% to 10% of the total mass of the chewable composition 13.

In addition to the mint and bedewer, the chewable composition 13 utilizes quantities of cannabis isolates. These isolates are concentrated forms of tetrahydrocannabinol and cannabidiols. The isolates can be combined with the other constituents of the chewable composition as a liquid or a solid. The liquid state of the tetrahydrocannabinol and cannabidiol isolates is an oil derived through solvent extraction and distillation. An example of a solvent utilized for this extraction is glycerin. The solid state of the tetrahydrocannabinol and cannabidiol isolates is crystalline and can be derived through a recrystallization extraction process. In the present embodiment, the tetrahydrocannabinol and cannabidiol isolates each comprise 1.4% of the total mass of the chewable composition 13. In other embodiments, the tetrahydrocannabinol and cannabidiol isolates each comprise an amount in the range of 1% to 10% of the total mass of the chewable composition 13.

To add sweetness to the chewable composition 13, a quantity of stevia extract is used in the chewable composition 13. Here, stevia extract refers to an extract of Stevia rebaudiana. This extract can be obtained through an aqueous extraction of the leaves of the stevia plant. In the present embodiment, stevia extract comprises 27.4 percent of the total mass of the chewable composition. In other embodiments, the stevia extract comprises an amount in the range of 20% to 40% of the total mass of the chewable composition 13.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A chewable composition and delivery pouch, comprising:
a chewable delivery pouch;
a chewable composition contained within the chewable pouch;
wherein the chewable composition comprises:
mint leaves;
a bedewer;
tetrahydrocannabinol isolate;
cannabidiol isolate;
stevia extract.

2) The chewable composition and delivery pouch of claim 1, wherein the mint leaves comprise 68.5 percent mass of the cannabis incorporated composition.

3) The chewable composition and delivery pouch of claim 1, wherein the bedewer is coconut oil.

4) The chewable composition and delivery pouch of claim 1, wherein the bedewer comprises 1.4 percent mass of the cannabis incorporated composition.

5) The chewable composition and delivery pouch of claim 1, wherein the tetrahydrocannabinol isolate comprises 1.4 percent mass of the cannabis incorporated composition.

6) The chewable composition and delivery pouch of claim 1, wherein the cannabidiol isolate comprises 1.4 percent mass of the cannabis incorporated composition.

7) The chewable composition and delivery pouch of claim 1, wherein the stevia extract comprises 27.4 percent mass of the cannabis incorporated composition.

8) The chewable composition and delivery pouch of claim 1, wherein the mint is chosen from the group of spearmint or peppermint.

9) The chewable composition and delivery pouch of claim 1, wherein the tetrahydrocannabinol isolate is an oil.

10) The chewable composition and delivery pouch of claim 1, wherein the tetrahydrocannabinol isolate is a crystalline isolate.

11) The chewable composition and delivery pouch of claim 1, wherein the cannabidiol isolate is an oil.

12) The chewable composition and delivery pouch of claim 1, wherein the cannabidiol isolate is a crystalline isolate.

13) The chewable composition and delivery pouch of claim 1, wherein the chewable composition comprises:
50 to 90 percent mass mint leaves;
1 to 10 percent mass bedewer;
1 to 10 percent mass tetrahydrocannabinol isolate;
1 to 10 percent mass cannabidiol isolate;
20 to 40 percent mass stevia extract.

14) A chewable composition and delivery pouch, comprising:
a chewable delivery pouch;
a chewable composition contained within the chewable pouch;
wherein the chewable composition consists essentially of:
68.5 percent mass mint leaves;
1.4 percent mass of a bedewer;
1.4 percent mass of a tetrahydrocannabinol isolate;
1.4 percent mass of a cannabidiol isolate;
27.4 percent mass stevia extract.

15) A chewable composition adapted to provide therapeutic benefits of Cannabis saliva with a breath freshener and delivery pouch, comprising:
a chewable delivery pouch;
a chewable composition contained within the chewable pouch;
wherein the chewable composition consists essentially of:
68.5 percent mass mint leaves;
1.4 percent mass of a bedewer;
1.4 percent mass of a tetrahydrocannabinol isolate;
1.4 percent mass of a cannabidiol isolate;
27.4 percent mass stevia extract.
